(19) United States
(12) Patent Application Publication
(10) Pub. No.: US 2003/0054988 A1
(43) Pub. Date: Mar. 20, 2003

(54) Modified plasminogen related peptide fragments and their use as angiogenesis inhibitors

(76) Inventors: Weidong-Richard Ji, Philadelphia, PA (US); Chester A. Meyers, Medford, NJ (US); Sesha I. Natarajan, Hillsborough, NJ (US); Pamela A. Trail, Madison, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/999,457

(22) Filed: Oct. 31, 2001

Related U.S. Application Data
(60) Provisional application No. 60/245,384, filed on Nov. 2, 2000.

(51) Int. Cl. A61K 38/49; C12N 9/68

(52) U.S. Cl. 514/12; 530/350; 435/217

(57) ABSTRACT

Modified peptide fragments of plasminogen domain are provided which exhibit anti-angiogenic activity. Compositions containing these peptide fragments and methods of using these compositions to treat angiogenic dependent and associated disorders are also provided.

### RELATED APPLICATIONS

This application claims priority benefit under Title 35 § 119(e) of U.S. provisional Application No. 60/245,384, filed Nov. 2, 2000.

### FIELD OF THE INVENTION

The present invention relates to peptide fragments of plasminogen and their use as inhibitors of angiogenesis. More specifically, the present invention relates to peptide fragments of plasminogen which have been modified to ensure linear conformation. The modified peptide fragments of the present invention are active in in vitro and in vivo assays of angiogenesis. Thus, these modified peptide fragments are believed to be useful as angiogenesis inhibitors in the treatment of cancer and other angiogenesis-related diseases.

### BACKGROUND OF THE INVENTION

Angiogenesis, as used herein, means the sprouting of new blood vessels from pre-existing blood vessels. Under normal physiological conditions, humans and other animals undergo angiogenesis in very restricted situations only. For example, angiogenesis is normally observed in wound healing, fetal and embryonic development and formation of the corpus luteum, endometrium and placenta.

Angiogenesis begins with the erosion of the basement membrane by enzymes released by endothelial cells and leukocytes. The endothelial cells, which line the lumen of blood vessels, then protrude through the basement membrane. Angiogenic stimulants induce the protruding endothelial cells to migrate through the eroded basement membrane and form, what is often referred to as, a sprout off of the parent blood vessel. These endothelial cells undergo mitosis and proliferation and the sprouts merge with each other to form capillary loops, thereby creating new blood vessels.

Pathological angiogenesis occurs in a number of diseases including, but not limited to, primary and metastatic tumor expansion and abnormal growth by endothelial cells, and supports the pathological damage associated with these diseases. The pathological diseases in which abnormal angiogenesis is present have been grouped into two categories, angiogenic dependent and angiogenic associated disorders. A review of angiogenesis and its relation to tumor growth is provided in PCT publication WO 95/29242.

Angiogenesis is tightly regulated by both positive and negative signals.

Positive signals of angiogenesis such as fibroblast growth factor (FGF) and vascular endothelial growth factor (VEGF) are potent mitogens for endothelial cell proliferation and strong chemoattractants for endothelial cell migration. These positive signals have been shown to promote neovascularization supportive of the expansion of both primary and metastatic tumors (Gross, J. L. et al. J. Natl Cancer Inst. 85(2):121-131; Kin et al. Nature 1993 3362(6243):841-844).

Angiostatin has been identified as one of the most effective negative signals of angiogenesis markedly suppressing the growth of a variety of tumors including carcinoma of the lung, prostate, colon and breast (O'Reilly et al. Cell 1994 79:315-328; O'Reilly et al. Nat. Med. 1996 2:689-692; Wu et al. Biochem. Biophys. Res. Commun. 1997 236:651-654). Angiostatin comprises an internal fragment of plasminogen and consists of three or four triple-looped kringle domains constrained by three disulfide bonds.

The various kringle domains of plasminogen share high sequence homology and structural similarity with each other and angiostatin. Like angiostatin, plasminogen domains such as kringle 5 (K5) have been reported to potently block both endothelial cell proliferation and migration, two steps that play pivotal roles in angiogenesis. Accordingly, various attempts have been made to use plasminogen domains or fragments thereof to modulate angiogenesis.

For example, U.S. Pat. No. 5,801,146 discloses use of a compound, mammalian kringle 5, for detection and treatment of diseases mediated by or associated with angiogenesis. This patent defines kringle 5 as a protein with three disulphide linkages, a molecular weight of approximately 9,000 to 11,0000 daltons as determined by reducing polyacrylamide gel electrophoresis, and an amino acid sequence substantially similar to a human plasminogen fragment beginning at about amino acid position 443 of an intact human plasminogen molecule.

Ji et al. (Biochem. Biophys. Res. Comm. 1998 247:414-419) describe the ability of recombinant kringle 5 of human plasminogen to inhibit endothelial cell migration. Experiments by Ji et al. showed reduced kringle 5 to display significantly increased anti-migratory activity as compared to its native form. From these studies it is suggested that the kringle conformation may shield kringle 5 from effective interaction with endothelial cells.

U.S. Pat. No. 6,057,122, 5,972,896 and 5,981,484 and PCT Application WO 97/41824 disclose mammalian kringle 5 peptide compounds for use in treating angiogenic diseases. These patents describe kringle 5 peptide compounds represented by the structural formula A-B-C-X-Y wherein A is absent or a nitrogen protecting group; Y is absent or a carboxylic acid protecting group; B is absent or is from 1 to about 197 naturally-occurring amino acid residues corresponding to amino acids 334 to 530 of human plasminogen; C is R¹-R²-R³-R⁴ wherein R¹ is lysyl, R² is leucyl or arginyl, R³ is tyrosyl, 3-I-tyrosyl or phenylalanyl, and R⁴ is aspartyl; and X is absent or from 1 to about 12 naturally occurring amino acids corresponding to amino acid 553 to 546 of human plasminogen. Also described in U.S. Pat. No. 6,057,122 are kringle 5 peptide compounds represented by the structural formula A-B₁-C₁-X₁-Y wherein A is absent or a nitrogen protecting group; Y is absent or a carboxylic acid protecting group; B₁ is absent or is from 1 to about 176 naturally occurring amino acid residues corresponding to amino acids 334 to 513 of human plasminogen; C₁ is the sequence from amino acid 514 to 523 of human plasminogen; and X₁ is absent or is from 1 to about 10 naturally occurring amino acid residues corresponding to the sequence from amino acid position 524 to amino acid position 533 of human plasminogen or analogues thereof.

### SUMMARY OF THE INVENTION

An object of the present invention is to provide peptide fragments of plasminogen which have been modified to ensure a linear conformation of the peptide fragment. Another object of the present invention is to provide compositions for inhibition of angiogenesis which comprise a peptide fragment of plasminogen which has been modified to ensure a linear conformation of the peptide fragment.

Another object of the present invention is to provide a method for inhibiting angiogenesis with a peptide fragment of plasminogen which has been modified to ensure a linear conformation of the peptide fragment.

Another object of the present invention is to provide a method for treating an angiogenic dependent or associated disorder in a subject which comprises administering to the subject a peptide fragment of plasminogen which has been modified to ensure a linear conformation of the peptide fragment.

Another object of the present invention is to provide a method for identifying potential inhibitors of angiogenesis which comprises identifying peptide fragments of plasminogen with a linear conformation and which exhibit anti-angiogenic activity in in vitro migration and/or proliferation assays of in vivo angiogenesis assays.

### DETAILED DESCRIPTION OF THE INVENTION

Various kringle domains of plasminogen and peptide fragments of these domains selectively antagonize endothelial cell growth and chemotaxis, thus indicating these domains to have an important role in regulating angiogenesis. It has now been found that plasminogen peptide fragments with a linear conformation exhibit enhanced anti-migratory activity as compared to the native form. Thus, plasminogen peptide fragments modified or selected to ensure a linear conformation can effectively interact with endothelial cells to suppress angiogenesis. Further, as demonstrated herein plasminogen peptide fragments modified to ensure a linear conformation also exhibited high potency in assays for angiogenesis.

The present invention relates to modified peptide fragments of plasminogen with a linear conformation and the use of these modified peptide fragments in inhibiting angiogenesis. For purposes of the present invention by “modified peptide fragment” it is meant a contiguous amino acid sequence based on the native amino acid sequence for plasminogen which is modified to ensure linear conformation. Various means for modifying a peptide fragment to ensure a linear conformation are known. Examples include, but are not limited to, replacement of cysteine residues in the fragment with alanine residues to prevent disulphide bond formation and reduction of the peptide fragment with dithiothreitol following by alkylation with iodoacetamide (Ji et al. Biochem. Biophys. Res. Comm. 1998 247:414-419). Thus, the modified peptide fragments of the present invention may comprise a contiguous amino acid sequence identical to a portion of a plasminogen domain or a variant thereof. By “variants” it is meant amino acid sequences with deletions, additions or amino acid substitutions as compared to the native plasminogen sequence which are also demonstrated to have anti-angiogenic activity in in vitro migration and/or proliferation assays of in vivo angiogenesis assays. By substitutions it is meant not only the replacement of cysteines with alanines to ensure a linear conformation, but also conservative amino acid substitutions such as replacement, one for another, of the aliphatic amino acids such as Ala, Val, Leu and Ile, the hydroxyl residues Ser and Thr, the acidic residues Asp and Glu, and the amide residues Asn and Gln. Peptide fragments of the present invention range in length between 1 and 79 amino acids, preferably 2 to 50 amino acids, and more preferably 4-20 amino acids.

A series of modified peptide fragments 16 to 32 amino acid residues in length representing discrete sections within plasminogen were prepared. To ensure linear conformation, the cysteine residues on each fragment were replaced by alanine residues to prevent disulfide bond formation. The peptide fragments synthesized for these experiments are depicted in Table 1.

TABLE 1
Sequences of Plasminogen Peptide Fragments

| | | SEQ ID. |
|---|---|---|
| Peptides | Peptide Sequence | NO: |
| BMS-291101 | Ac-AMFGNGKGYRGKRATT-NH₂ | 1 |
| BMS-291102 | Ac-VTGTPAQDWAAQEPHR-NH₂ | 2 |
| BMS-291201 | Ac-HSIFTPETNPRAGLEK-NH₂ | 3 |
| BMS-291203 | Ac-NYARNPDGDVGGPWAY-NH₂ | 4 |
| BMS-291205 | Ac-TTNPRKLYDYADVPQA-NH₂ | 5 |
| BMS-291206 | Ac-TTNPRKRYDYADVPQA-NH₂ | 6 |
| BMS-291208 | Ac-YRGKRATTVTGTPAQD-NH₂ | 7 |
| BMS-291209 | Ac-WAAQEPHRHSIFTPET-NH₂ | 8 |
| BMS-219211 | Ac-NPRAGLEKNYARNPDG-NH₂ | 9 |
| BMS-291221 | Ac-DVGGPWAYTTNPRKLY-NH₂ | 10 |
| BMS-291241 | Ac-GKRATTVTGTPAQDWAAQEPHRHSIFT | 11 |
| | PET-NH₂ | |
| BMS-291242 | Ac-AQDWAAQEPHRHSIFTPETNPRAGLEK | 12 |
| | NYA-NH₂ | |
| BMS-291245 | Ac-NYARNPDGDVGGPWAYTTNPRKLYDYA | 13 |
| | DVPQA-NH₂ | |

Modified peptide fragments of the present invention, as exemplified in Table 1, were shown to inhibit bFGF- and/or VEGF-elicited human umbilical vein endothelial cell (HUVEC) migration. The assay used in these experiments is described by Ji et al. in Biochem. Biophys. Res. Commun. 1998 247:414-419. These experiments demonstrated that a group of modified peptide fragments including BMS-291102 (SEQ ID NO: 2), BMS-291203 (SEQ ID NO: 4), BMS-291206 (SEQ ID NO: 6), and BMS-291245 (SEQ ID NO: 13) were extremely potent (IC₅₀<10 nm) or potent (IC₅₀<100 nm) inhibitors of both bFGF- and VEGF-induced HUVEC migration. The modified peptide fragment BMS-291205 (SEQ ID NO: 5) was also a potent inhibitor of VEGF-stimulated HUVEC migration and exhibited marginal inhibitory activity (IC₅₀=1000 nm). In addition, the modified peptide fragment BMS-291242 (SEQ ID NO: 12) was an extremely potent inhibitor of bFGF-induced HUVEC migration.

The anti-proliferative effects of the modified peptide fragments on endothelial cells in response to different mitogenic stimulation were also examined. The assay used in these experiments is described by Cao et al. in J. Biol. Chem. 1997 272(36):22924-22928. The modified peptide fragment BMS-291102 (SEQ ID NO: 2) was an extremely potent inhibitor of bFGF-elicited HUVEC proliferation. The modified peptide fragment BMS-291242 (SEQ ID NO: 12) also exhibited moderate anti-proliferative activity.

Modified peptide fragments of the present invention were also evaluated in the matrigel plug model to determine their in vivo anti-angiogenic activities. To minimize the effects caused by differential pharmacokinetic properties of these compounds, the matrigel was prepared together with these peptides and the mixture was implanted into nude mice. At the dose of 100 μM/plug, modified peptide fragments BMS-291102 (SEQ ID NO: 2) and BMS-291203 (SEQ ID NO: 4) effectively reduced the number of endothelial cells in the matrigel plug in response to bFGF-stimulation in a dose-dependent fashion. Other modified peptide fragments of the present invention which significantly inhibited bFGF-elicited in vivo angiogenesis at the dose of 100 μM/plug included BMS-291242 (SEQ ID NO: 12) and BMS-291245 (SEQ ID NO: 13).

The present invention thus provides compositions comprising peptide fragments of plasminogen which have been modified to ensure a linear conformation for use in inhibiting angiogenesis. Compositions of the present invention preferably comprise the modified peptide fragment and a pharmaceutically acceptable vehicle. Preferred modified peptide fragments for incorporation into compositions of the present invention are those exhibiting anti-angiogenic activity either in in vitro migration and/or proliferation assays or in vivo angiogenesis assays. Examples of preferred modified peptide fragments include, but are not limited to, BMS-291102 (SEQ ID NO: 2), BMS-291203 (SEQ ID NO: 4), BMS-291205 (SEQ ID NO: 5), BMS-291206 (SEQ ID NO: 6), BMS-291242 (SEQ ID NO: 12) and BMS-291245 (SEQ ID NO: 13). Further, it is expected that variants of these modified peptide fragments or other modified peptide fragments overlapping in amino acid sequence with these preferred peptide fragments will exhibit similar anti-angiogenic activity. Such modified peptide fragments can be routinely synthesized and screened for anti-angiogenic activity in accordance with methods described herein and thus are included within the scope of the present invention.

The present invention also provides a method for identifying peptide fragments in the native form of plasminogen sequences as potential inhibitors of angiogenesis. As exemplified by BMS-291209 (see Table 1), some peptide fragments of plasminogen maintain a linear conformation naturally without modification. The linear peptide fragment BMS-291209 exhibited extremely potent inhibitory activity of VEGF-induced migration of HUVEC cells and potent inhibitory activity of bFGF-induced migration of HUVEC cells. Accordingly, it is expected that other plasminogen peptide fragments which maintain a linear conformation naturally will exhibit similar anti-angiogenic activities. Thus, peptide fragments of plasminogen with a naturally linear conformation can be identified and screened for anti-angiogenic activity in in vitro migration and/or proliferation assays of in vivo angiogenesis assays to rationally identify inhibitors of angiogenesis.

Plasminogen peptide fragments of the present invention can be prepared synthetically using an automated peptide synthesizer or recombinantly. Various methods for such syntheses are well known to those of skill in the art.

Pharmaceutically acceptable vehicles useful in the present invention may comprise a carrier, adjuvant or vehicle that can be administered to a subject, incorporated into a composition of the present invention, and which do not destroy the pharmacologic activity thereof. Examples of pharmaceutical vehicles useful in the present invention include, but are not limited to, ion exchangers, alumina, aluminum stearate, lecithin, self-emulsifying drug delivery systems such as d(-tocopherol polyethyleneglycol 1000 succinate, surfactants used in pharmaceutical dosage forms such as TWEENS and other similar polymeric delivery matrices, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethocellulose, polyacrylates, waxes, polyethylene-polyoxypropylene-block polymers, and wool fat. Cyclodextrins such as α-, β- and γ-cyclodextrin, or chemically modified derivatives such as hydroxyalkylcyclodextrins, including 2- and 3-hydroxypropyl-β-cyclodextrins, or other solubilized derivatives can also be used to enhance delivery of the compositions of the present invention.

The compositions of the present invention may further comprise additional therapeutic agents useful in the treatment of angiogenic dependent and/or associated disorders. Examples of additional agents useful in the present invention include, but are not limited to antiinflammatories, antiproliferatives, chemotherapeutic agents, immunosuppressants and antimicrobials. Amounts of additional agents to be included within the compositions of the present invention can be determined routinely by those of skill in the art based upon standard text references such as the Physician's Desk Reference.

Various pharmaceutical formulations comprising compositions of the present invention can be prepared routinely by those of skill in the art using conventional solid or liquid vehicles or diluents, as well as pharmaceutical additives, selected in accordance with the desired mode of administration.

Compositions of the present invention can be administered by any suitable means, for example orally, such as in the form of tablets, capsules, granules or powders; sublingually; bucally; parenterally, such as by subcutaneous, intracutaneous, intravenous, intramuscular, intraarticular, intraarterial, intrasynovial, or intasternal, intrathecal, intralesional and intracranial injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic pharmaceutically acceptable vehicles. The compositions of the present invention can be administered in a form suitable for immediate release. Alternatively, an extended release formulation can also be used. Compositions of the present invention can also be administered liposomally.

Exemplary compositions for oral administration include: suspensions which may contain, for example, microcrystalline cellulose for impairing bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binder, extenders, disintegrants, diluents and lubricants known in the art. Compositions of the present invention can also be delivered sublingually or bucally through the oral cavity via, for example, molded tablets, compressed tablets or freeze-dried tablets. Examples of fast dissolving diluents for use in these formulations include, but are not limited to, mannitol, lactose, sucrose and/or cyclodextrins. Such formulations may further comprise high molecular weight excipients such as celluloses (avicel) or polyethylene glycol. Excipients to aid in mucosal adhesion such as hydroxypropylcellulose, hydroxypropylmethylcellulose, sodium carboxymethyl cellulose, maleic anhydride copolymer and agents to control release such as polyacrylic copolymer can also be incorporated into these formulations. In addition, the formulations may comprise lubricants, glidants, flavors, coloring agents and stabilizers which ease fabrication and use.

Exemplary compositions for nasal aerosol or inhalation administration include solutions in saline. These solutions may also contain preservatives such as benzyl alcohol, absorption promoters to enhance bioavailability and/or solubilizing or dispersing agents.

Exemplary compositions for parenteral administration include injectable solutions or suspensions which may contain, for example, suitable non-toxic parenterally acceptable diluents or solvents such as mannitol, 1,3-butanediol, water, Rhinger's solution, an isotonic sodium chloride solution, or other suitable dispersing or wetting and suspending agents including synthetic mono- or di-glycerides and fatty acids such as oleic acid.

Exemplary compositions for rectal administration include suppositories which may contain, for example, a suitable non-irritating excipient such as cocoa butter, synthetic glyceride esters or polyethylene glycols, which are solid at room temperature, but which liquefy and/or dissolve in the rectal cavity to release the active compound.

Exemplary compositions for topical administration include a topical carrier such as PLASTIBASE (mineral oil gelled with polyethylene)

Compositions of the present invention comprising a modified peptide fragment are useful in treating angiogenic dependent and/or associated disorders. Thus, the present invention also relates to method of treating an angiogenic dependent or associated disorder with an effective amount of a composition comprising a modified peptide fragment of the present invention. Angiogenesis dependent and associated diseases which can be treated include, but are not limited to: solid tumors; blood borne tumors such as leukemias; tumor metastasis; benign tumors such as hemangiomas, acoustic acuromas, neurofibromas, trachomas, and pyogenic granulomas; rheumatoid arthritic; psoriasis; ocular angiogenic diseases such as diabetic retinopathy, retinopathy of prematurity, macular degeneration, corneal graft rejection, neovascular glaucoma, retrolental fibroplasia, and rubeosis; Osler-Webber Syndrome; myocardial angiogenesis; plaque neovascularization; telangiectasia; hemophiliac joints; angiofibroma; and wound granulations.

Compositions of the present invention are also useful in treating diseases or conditions of excessive or abnormal stimulation of endothelial cells. Examples of these disorders includes, but are not limited to, intestinal adhesions, atherosclerosis, scleroderma, and hypertrophic scars such as keloids.

These compositions are also useful as birth control agents via prevention of the vascularization required for embryo implantations.

The compositions and methods of the present invention can also be used in combination with other compositions or procedures for the treatment of angiogenic dependent and associated disorders. For example, in one embodiment, these compositions are used in combination with surgery, radiation or chemotherapy, to treat tumors. It is believed that co-administration of the compositions of the present invention extends the dormancy of micrometastasis and stabilizes any residual primary tumor.

The effective amount of a composition comprising a modified peptide fragment to be administered to a subject can be determined routinely by one of skill in the art based upon in vitro and in vivo assays such as described herein. Exemplary dosage amounts for an adult human typically range from about 0.1 to 500 mg/kg of body weight of the modified peptide fragment per day. Compositions of the present invention can be administered as a single dose, in the form of individual divided doses, such as from 1 to 5 times per day, or as a sustained release formulation over an extended period of 24 hours or more. As will be understood by those of skill upon reading this disclosure, the specific dose level and frequency of dosage for a particular subject may be varied and will depend upon a variety of factors including the activity of the specific compound employed, the metabolic stability and length of action of that compound employed, the species, age, body weight, general health, sex and diet of the subject, the mode and time of administration, rate of excretion and clearance, drug combination, and severity of the particular conditions. Preferred subjects for treatment include animals, more preferably mammalian species such as humans, and domestic animals such as dogs, cats and the like, which are subject to angiogenic dependent and/or associated diseases.

The following nonlimiting examples are provided to further illustrate the present invention.

### EXAMPLES

### Example 1

### Peptide Synthesis

Ten of the 14 plasminogen peptide fragments were initially prepared by simultaneous automated solid phase synthesis on an Advanced Chemtech Model 396 “Multiple Peptide Synthesizer” (MPS) using standard Fmoc chemistry protocols. Global removal of protecting groups and cleavage from the solid support was performed in one step by standard methods. Purification of final products was accomplished by preparative RP-HPLC. The other four peptides were prepared individually on an Applied Biosystems Model 433A peptide synthesizer using Fmoc/HBTU protocols. Resin cleavage and deprotection was as above. For the cyclic peptide, intramolecular closure of the disulfide bond was accomplished by air oxidation in dilute aqueous solution at pH 8.5 and monitored for completion by analytical RP-HPLC. Final purifications were by preparative RP-HPLC.

### Example 2

### In vivo Matrigel Angiogenesis Model

MATRIGEL was mixed with basic fibroblast growth factors at a final concentration of 300 μg/ml. Peptide fragments at selected doses were embedded with the MATRIGEL mixture and injected subcutaneously into mice. The animals were allowed to grow for 7 days. The plugs were then excised and the number of endothelial cells in the plug were examined histologically.

What is claimed is:

1. A peptide fragment of plasminogen which has been modified to ensure a linear conformation of the peptide fragment.

2. The modified peptide fragment of claim 1 comprising SEQ ID NO: 1, 2,4,5,6,7,9, 10, 11, 12 or 13.

3. A composition for inhibition of angiogenesis comprising a modified peptide fragment of claim 1 which exhibits anti-angiogenic activity and a pharmaceutically acceptable vehicle.

4. The composition of claim 3 wherein the modified peptide fragment comprises SEQ ID NO: 2, 4, 5, 6, 12 or 13.

5. A method for inhibiting angiogenesis which comprises contacting cells with a modified peptide fragment of claim 1.

6. A method for treating an angiogenic dependent and associated disorder in a subject comprising administering to the subject an effective amount of a composition of claim 3.

7. The method of claim 6 wherein the angiogenic dependent and associated disorder comprises a solid tumor, a blood borne tumor, tumor metastasis, a benign tumor, rheumatoid arthritis, psoriasis, an ocular angiogenic disease, Osler-Webber Syndrome, myocardial angiogenesis, plaque neovascularization, telangiectasia, hemophiliac joints, angiofibroma, or wound granulations.

8. A method for treating a disease or condition of excessive or abnormal stimulation of endothelial cells in a subject comprising administering to the subject an effective amount of a composition of claim 3.

9. The method of claim 8 wherein the disease or conditions comprises intestinal adhesions, atherosclerosis, scleroderma or hypertrophic scars.

10. A method for identifying potential inhibitors of angiogenesis comprising identifying peptide fragments of plasminogen with a linear conformation and assessing anti-angiogenic activity of the linear fragment.


SEQUENCE LISTING (13 sequences)

SEQ ID NO: 1 - protein, 16 aa, Artificial Sequence
  Artificial Sequence = Homo Sapien
AMFGNGKGYRGKRATT

SEQ ID NO: 2 - protein, 16 aa, Artificial Sequence
  Artificial Sequence = Homo Sapien
VTGTPAQDWAAQEPHR

SEQ ID NO: 3 - protein, 16 aa, Artificial Sequence
  Artificial Sequence = Homo Sapien
HSIFTPETNPRAGLEK

SEQ ID NO: 4 - protein, 16 aa, Artificial Sequence
  Artificial Sequence = Homo Sapien
NYARNPDGDVGGPWAY

SEQ ID NO: 5 - protein, 16 aa, Artificial Sequence
  Artificial Sequence = Homo Sapien
TTNPRKLYDYADVPQA

SEQ ID NO: 6 - protein, 16 aa, Artificial Sequence
  Articial Sequence = Homo Sapien
TTNPRKRYDYADVPQA

SEQ ID NO: 7 - protein, 16 aa, Artificial Sequence
  Artificial Sequence = Homo Sapien
YRGKRATTVTGTPAQD

SEQ ID NO: 8 - protein, 16 aa, Artificial Sequence
  Artificial Sequence = Homo Sapien
WAAQEPHRHSIFTPET

SEQ ID NO: 9 - protein, 16 aa, Artificial Sequence
  Artificial Sequence = Homo Sapien
NPRAGLEKNYARNPDG

SEQ ID NO: 10 - protein, 16 aa, Artificial Sequence
  Artificial Sequence = Homo Sapien
DVGGPWAYTTNPRKLY

SEQ ID NO: 11 - protein, 30 aa, Artificial Sequence
  Artificial Sequence = Homo Sapien
GKRATTVTGTPAQDWAAQEPHRHSIFTPET

SEQ ID NO: 12 - protein, 30 aa, Artificial Sequence
  Artificial Sequence = Homo Sapien
AQDWAAQEPHRHSIFTPETNPRAGLEKNYA

SEQ ID NO: 13 - protein, 32 aa, Artificial Sequence
  Artificial Sequence = Homo Sapien
NYARNPDGDVGGPWAYTTNPRKLYDYADVPQA